(19) United States
(12) Patent Application Publication
(10) Pub. No.: US 2026/0165621 A1
(43) Pub. Date: Jun. 18, 2026

(54) SYSTEM AND SKIN-BASED SENSOR PLATFORM TO MONITOR BIOCHEMICAL ACTIVITY OF A WEARER

(71) Applicant: Accenture Global Solution Limited, Dublin (IE)

(72) Inventors: Lavinia Andreea DANIELESCU, Seattle, WA (US); Eric Michael Gallo, Moretown, VT (US); Luke Fabrice Gockowski, Oakland, CA (US); Wade Ingram, San Francisco, CA (US); JungWook Park, Sunnyvale, CA (US)

(21) Appl. No.: 18/980,350

(22) Filed: Dec. 13, 2024

(51) Int. Cl. A61B 5/1486; A61B 5/00; A61B 5/1455

(52) U.S. Cl. CPC A61B 5/14865; A61B 5/0002; A61B 5/1455; A61B 5/6833; A61B 5/6804; A61B 2562/12

(57) ABSTRACT

Implementations of the present disclosure discloses a system and skin-based sensor platform to monitor biochemical activity of a wearer. The system includes an apparatus for monitoring biochemical activity of a wearer, wherein the apparatus includes a base substrate layer including a carbon-based material having one or more laser induced graphene (LIG) fabricated elements. The one or more LIG fabricated elements include an inductor-capacitor circuit to receive a wireless signal and to provide passive wireless readouts, and one or more sensing circuits electrically coupled to the inductor-capacitor circuit to detect biochemical activity associated with the wearer of the apparatus. The apparatus further includes an adhesive layer provided on a first side of the base substrate layer to provide adhesion to the skin of the wearer, and an encapsulation layer placed on an opposing side of the base substrate layer. The system further includes, a transmitter receiver device, the transmitter receiver device including a transmitting controller to transmit the wireless signals and receive data associated with the biochemical activity, data storage to store the data associated with the biochemical activity, and a battery to provide and regulate power for the biochemical sensor system.

### FIELD OF THE INVENTION

Various embodiments described herein relate generally to a technique for sustainably monitoring biochemical activity of a wearer. More particularly, the present disclosure relates to an apparatus and system for monitoring biochemical activity of a wearer and a method to fabricate the apparatus.

### BACKGROUND

With ever-increasing stress levels and lifestyle changes, there is an increased demand to provide continuous health monitoring devices while reducing the environmental impact of conventional disposable monitoring devices. Moreover, there is need to provide real-time feedback and timely reminders to a wearer or a user regarding health monitoring. Such monitoring devices should be lightweight, portable, and preferably should not create pollution upon disposal, for example, contributing to landfill. Therefore, there is a need for a device that continuously monitors the health of the wearer, which is unobtrusive, and is preferably biodegradable. Such devices may be worn on the skin of a wearer and/or integrated with the skin of a wearer for skin surface and/or subcutaneous sensing. For specific health conditions such a device may be configured to monitor relevant biochemical activity, biomarkers, and/or organic processes. In using sustainable biocompatible materials, biodegradable monitoring devices offer a promising solution for various applications, including healthcare, diagnostics, environmental monitoring, etc.

### SUMMARY

Implementations of the present disclosure are generally directed to a system and a technique for skin-based monitoring of a wearer's health. More particularly, the present disclosure relates to an apparatus and a system for monitoring biochemical activities of a wearer and a method to fabricate such a device.

In general, innovative aspects of the subject matter described in this specification provide an apparatus and a system for monitoring biochemical activity of the wearer and a method for fabricating such an apparatus. The wearer of the apparatus may include, for example, a human being, an animal, and a pet, etc. The device includes laser induced graphene (LIG) fabricated elements on a base substrate layer. The LIG fabricated elements include an inductor-capacitor circuit to receive a wireless signal and to provide passive wireless readouts, sensing circuits to detect biochemical activity associated with the wearer of the apparatus which are electrically coupled to the inductor-capacitor circuit. The apparatus includes an adhesive layer on a side of the base substrate layer to provide attachment to the skin of the wearer. Additionally, the apparatus has a coating of an encapsulating material on an opposing side of the base substrate layer to form a protective layer, wherein said encapsulation material may be water resistant and/or hydrophobic.

It is appreciated that the apparatus in accordance with the present disclosure can include any combination of the aspects and features described herein. That is, the system in accordance with the present disclosure are not limited to the combinations of aspects and features specifically described herein, but also include any combination of the aspects and features provided.

The details of one or more implementations of the present disclosure are set forth in the accompanying drawings and the description below. Other features and advantages of the present disclosure will be apparent from the description and drawings, and from the claims.

### BRIEF DESCRIPTION OF DRAWINGS

Various embodiments in accordance with the present disclosure will be described with reference to the drawings, in which:

FIG. 1 illustrates a sensor platform in accordance with implementations of the present disclosure;

FIG. 2 illustrates an exemplary fabrication technique for the sensor platform in accordance with implementations of the present disclosure;

FIG. 3 illustrates an exemplary embodiment of a sensor platform including a subcutaneous sensing element, in accordance with implementations of the present disclosure;

FIG. 4A illustrates an optical sensor, in accordance with implementations of the present disclosure;

FIG. 4B illustrates an optical sensor in the form of a stripe, in accordance with an embodiment of the present disclosure;

FIG. 4C illustrates an exemplary circuit of an optical sensor, in accordance with implementations of the present disclosure;

FIG. 5A illustrates an exemplary embodiment of a system for monitoring biochemical activity of a wearer, in accordance with implementations of the present disclosure;

FIG. 5B illustrates an exemplary embodiment of components of the biochemical sensor system, in accordance with implementations of the present disclosure;

FIG. 6 illustrates an example circuit including a receiving coupling inductor and passive components, in accordance with implementations of the present disclosure;

FIG. 7 illustrates an example disclosing a wireless power and readout process, in accordance with implementations of the present disclosure;

FIG. 8 is a flow diagram that represents an example method for fabricating an apparatus;

FIG. 9 is a flow diagram that represents an example method for fabricating an apparatus having optical devices; and

FIG. 10 illustrates a block diagram of an example environment in accordance with implementations of the present disclosure.

Like reference numbers and designations in the various drawings indicate like elements.

### DETAILED DESCRIPTION OF DRAWINGS

In the following description, various embodiments will be illustrated by way of example and not by way of limitation in the figures of the accompanying drawings. References to various embodiments in this disclosure are not necessarily to the same embodiment, and such references mean at least one. While specific implementations and other details are discussed, it is to be understood that this is done for illustrative purposes only. A person skilled in the relevant art will recognize that other components and configurations may be used without departing from the scope and spirit of the claimed subject matter.

Reference to any “example” herein (e.g., “for example”, “an example of”, by way of example” or the like) are to be considered non-limiting examples regardless of whether expressly stated or not.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any exemplified term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

The term “comprising” when utilized means “including, but not necessarily limited to”; it specifically indicates open-ended inclusion or membership in the so-described combination, group, series and the like.

The term “a” means “one or more” unless the context clearly indicates a single element.

“First,” “second,” etc., re labels to distinguish components or blocks of otherwise similar names but does not imply any sequence or numerical limitation.

“And/or” for two possibilities means either or both of the stated possibilities (“A and/or B” covers A alone, B alone, or both A and B take together), and when present with three or more stated possibilities means any individual possibility alone, all possibilities taken together, or some combination of possibilities that is less than all of the possibilities. The language in the format “at least one of A. and N” where A through N are possibilities means “and/or” for the stated possibilities (e.g., at least one A, at least one N, at least one A and at least one N, etc.).

It should also be noted that in some alternative implementations, the functions/acts noted may occur out of the order noted in the figures. For example, two steps disclosed or shown in succession may in fact be executed substantially concurrently or may sometimes be executed in the reverse order, depending upon the functionality/acts involved.

Specific details are provided in the following description to provide a thorough understanding of embodiments. However, it will be understood by one of ordinary skill in the art that embodiments may be practiced without these specific details. For example, systems may be shown in block diagrams so as not to obscure the embodiments in unnecessary detail. In other instances, well-known processes, structures, and techniques may be shown without unnecessary detail to avoid obscuring example embodiments.

The specification and drawings are to be regarded in an illustrative rather than a restrictive sense. It will, however, be evident that various modifications and changes may be made thereunto without departing from the broader spirit and scope of the invention as set forth in the claims.

Changes in lifestyles along with advances in medical science have created a demand for wearable, real-time health monitoring devices. Most of the existing devices are cumbersome and are not biodegradable which ultimately creates waste pollution in the environment, for example in landfills and the like, at the end of their use.

Accordingly, there is a need for a lightweight health monitoring device that benefits from being in direct contact with a wearer to provide regular and/or event-based updates about the wearer's physiology for example, cortisol levels as they vary in response to environmental stressors. The device should also be designed with end-of-life biodegradability in mind, for example, the device may be made from biodegradable materials that can decompose in tens to hundreds of days, rather than plastics which may take years or decades to degrade. Additionally, such devices may be powered externally to prevent the need for recharging or disposal of energy storage elements.

In view of this, some embodiments of the present disclosure propose a batteryless skin-worn sensor platform capable of providing real-time, continuous or periodic, updates on levels of biochemical activities of the wearer's body. The sensor platform may be biodegradable, sustainable, and eco-friendly. The sensor platform may be worn on the skin of the wearer for shorter durations in the range of hours to days, to longer durations in the range of days to weeks. The biochemical activities monitored by the sensor platform may include one or more of glucose, cortisol, sodium, lactate, or ethanol, etc. The sensor platform in the present disclosure may receive power from an external power source, for example, through inductively coupled power transfer from a transmitter.

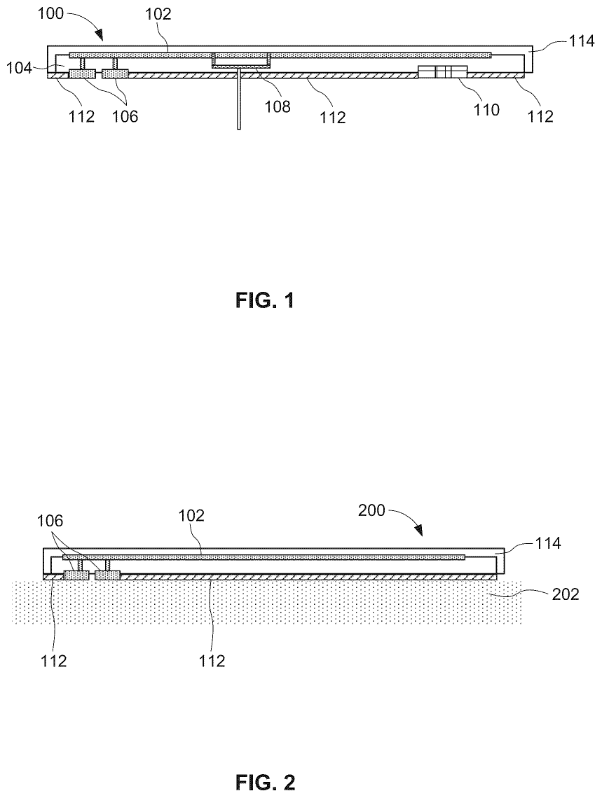

FIG. 1 illustrates a sensor platform 100, in accordance with implementations of the present disclosure.

The sensor platform 100 includes a base substrate layer 104 made of a carbon-based material with one or more laser induced graphene (LIG) fabricated elements 102 and 106. The LIG fabricated elements 102 and 106 may be created on the base substrate layer 104 through a controlled exposure to a focused or slightly defocused laser beam, enabling direct write formation on the surface of the substrate layer 104. The laser may remove non-carbon materials, breaking chemical bonds, and under the correct conditions the constituent carbon atoms of the base substrate layer 104 reform into an electrically conductive graphene material. Laser wavelengths between 1500 and 300 nm may be used at power levels tuned to a specific material of the base substrate layer 104 and desired utility of the LIG. Typically, power levels range from 100's mW to 10's W.

The one or more LIG fabricated elements 102 and 106 may include an inductor-capacitor circuit 102, to receive wireless power and signals and to provide passive wireless readouts to an external device and one or more LIG fabricated skin sensing elements 106 to detect biochemical activity of the wearer, wherein the LIG fabricated skin sensing elements 106 are electrically coupled to the inductor-capacitor circuit 102. The one or more LIG fabricated skin sensing elements 106, may be electrically passive and each LIG fabricated skin sensing element 106 may be configured to monitor biochemical activity of either glucose, cortisol, sodium, lactate, or ethanol, etc., of the wearer. For example, the sensor platform 100 may be configured to sense one or more of heart rate, cortisol levels, glucose levels, or other parameters. The one or more LIG fabricated skin sensing elements 106 may include a subcutaneous sensing element 108. The subcutaneous sensing element 108 may be made using a glucose-responsive fluorescence-based sensitive biodegradable material. The sensor platform 100 further includes an adhesive layer 112 on the wearer contact side of the base substrate layer 104 to provide adhesion to the skin of the wearer. Additionally, an encapsulation layer 114 is provided on an opposing side of the base substrate layer 104. The base substrate layer 104 may be a thin layer in the order of 0.1˜100 μm to ensure that the design of the sensor platform 100 mimics any mechanical properties of the skin of a wearer at a desired location. Further, starch-based adhesives or similar materials with a thickness in the order of 10s of microns may be used for the adhesion layer 112 to ensure that the sensor platform 100 retains a thin form factor. The thin form factor of the sensor platform 100 helps to reduce a need for strong adhesives and makes it more comfortable for the wearer. In a preferred embodiment, the base substrate layer 104, the adhesive layer 112 and the encapsulation layer 114, may be provided to ensure that a distance between a distal side of the adhesive layer 112 and a distal side of the encapsulation layer 114 may be 1 μm to 200 μm. Further, the base substrate layer 104, the one or more LIG fabricated skin sensing elements 106, including the subcutaneous sensing element 108, the adhesive layer 112 and the encapsulation layer 114 may be fabricated from biodegradable materials such as biomaterials.

In the present disclosure, biomaterials may be used in which, a first biomaterial may be used for forming the functional substrate layer 104, and a second biomaterial may be used for forming an encapsulation, protection, and/or waterproofing layer 114. The base substrate layer 104 may be a carbon-based body where lasing may induce electrically conductive graphene elements through a LIG process, to create contacts, sensors, traces and/or other electrical elements directly on the substrate layer 104. The base substrate layer 104 may be at least partially formed of at least one or more of bamboo, lignin, chitosan, starch, cellulose, etc., which may serve as precursors to the LIG elements 102 and 106. The LIG process may also be used to form or interconnect the electrically conductive graphene elements 114, 102, and further to form transistors, inductors, capacitors, resistors, which may be used as passive sensing elements. The LIG fabricated elements 102 and 106 may further include a separate reference circuit, which may include an inductor-capacitor circuit, to provide a static reference signal to compare with sensed signals. The encapsulation layer 114 may be water resistant and/or provide hydrophobic encapsulation, for example, the material of the encapsulation layer 114 may be fabricated from one or more of shellac, modified starch, modified cellulose, gelatin etc. Some additional materials may be used in the fabrication of the sensor platform 100, for example, PEDOT-PSS or other equivalent material may be used for a transistor gate, while other organic compounds may be used for optical components. Additionally, sustainable circuitry (ZnO, MgO or organic transistors) may be incorporated in the circuitry to provide signal gain or conditioning on the sensor platform 100, for accurate measurements. In some implementations, the base substrate layer 104 may further include one or more optical sensors 110 that may be used as a sensing element in the sensor platform 100. The one or more optical sensors 110 may be made of DNA-based mixtures or polymer LEDs (PLED) to create the emitting and electronic layers of the devices. Further, the one or more optical sensors 110 may be formed as emitter-detector rings or in the form of strips. In a preferred embodiment, the one or more optical sensors 110 are formed as emitter-detector rings, and the formation is described in detail further below in the present disclosure. Depending on an optimal detection technique, the one or more LIG fabricated elements 102 and 106 may be on a single side or use traces which wraparound the edges of the base substrate layer 104 or through-hole vias to connect the two sides of the base substrate layer 104. The material of the base substrate layer 104, the material of the encapsulation layer 114, the material of the adhesion layer 112, and the dimensions of the sensor platform 100, may be determined by the properties and mechanics of the wearer's skin in a desired attachment location to optimize adhesion. For example, the sensor platform 100 may be the size of a bandage, and/or compatible with a medical bandage.

The present disclosure discusses wireless techniques, for example, inductor-capacitor (LC) passive and wireless resistive passive readout techniques. A powered transmitter/receiver or coupling inductor circuit sends a wireless signal to the LIG passive circuitry of the sensor platform 100 at a specified frequency. This specific frequency is received at the passive LIG inductor-capacitor circuit 102 of the sensor platform 100, injecting power into the other passive LIG fabricated elements for example 106, causing the other passive LIG fabricated elements to oscillate, storing some energy, and causing the passive LIG inductor-capacitor circuit 102 to send a signal at its resonant frequency which is received by the powered transmitter/receiver or the coupling inductor circuit. An electronic state of the passive LIG fabricated elements in the sensor platform 100 is associated with the frequency of the return signal to the powered transmitter/receiver. The electronic state of the passive LIG fabricated elements in the sensor platform 100 being dependent on the LIG fabricated sensor elements, and hence determined by the biochemical activity of the wearer. Alternatively, sensor readings can be obtained by sweeping transmitted frequencies across a narrow range to identify the resonant frequency by locating the frequency at which the transmitter shows minimum impedance values. The LIG fabricated elements can further include a LIG fabricated reference receiver circuit to provide a static reference signal to compare with sensed signals. This static circuit may be included on the same substrate with no sensing elements to provide a reference signal to compare the sensing circuit response, accounting for changes to the substrate or non-sensing circuit elements, reduced signal strength, proximity or orientations changes between transmitter and receiver, temperature fluctuations, etc. It is important to note that the sensor platform 100 shown in FIG. 1 includes a single passive LIG inductor-capacitor circuit 102 which is powered using a single transmitter. However, the sensor platform 100 may include one or more passive LIG inductor-capacitor circuits and may be powered using one or more transmitters.

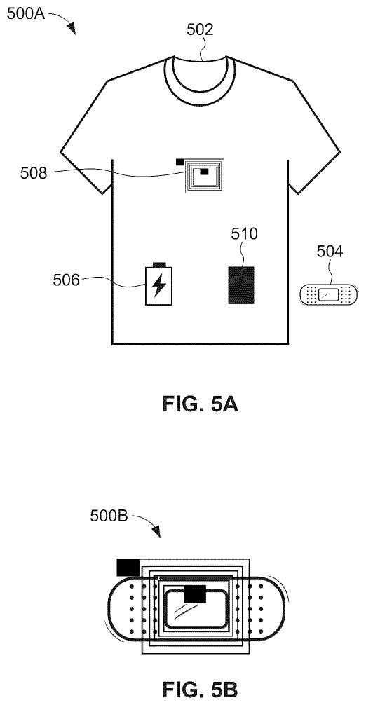

Biochemical activity monitoring may be performed through a functionalization of the LIG fabricated sensing elements 102 and 106 to detect targeted biochemicals in sweat and/or on the skin of a wearer. Additionally, subcutaneous sensing may be performed using a subcutaneous sensing element 108 with one more LIG fabricated elements used as electronic vias and traces to connect the sensing element 108 to the main circuitry of the sensor platform 100. The subcutaneous sensing element 108 may be biodegradable. The one or more LIG fabricated skin sensing elements 106 may be configured to implement resistive and/or capacitive sensing. The resistive sweat sensing element may be functionalized with additional chemicals to tune detection. For example, the sensing circuits may be coated with an enzymatic material to detect the biochemical activity which result in a change in the component value, resistance or capacitance. Some enzymatic materials may include, but are not limited to, glucose oxidase (GOx), glucose dehydrogenase (GDH), lactate oxidase, and ascorbate oxidase. In a preferred embodiment, Gox or GDH material is coated to the sensing circuits to detect biochemical activity. The one or more LIG fabricated skin sensing elements 106 may be configured to implement active sensing where a voltage is generated in response to the detected chemicals or biochemical activity. Biochemical activity can produce a voltage potential, for example but not limited to the glucose sensors. The generated potential can be used within the circuit to interact with elements, such as applying the potential to the base of a transistor which modifies the measured resistance across its emitter and collector. The LIG of the base substrate 104 may also provide electrical contacts to the one or more optical sensors 110 for optical sensing, using a combination of photoemitters and photodetectors utilizing suitable materials which may be biodegradable. A hand-held or textile-based wireless transmitter/receiver (shown in FIGS. 7 and 5A) may be used to power the sensor platform 100 and receive signals associated with the biochemical activity of the wearer. For example, using skin-compatible adhesion, the apparatus or a skin patch is attached to an arm and is then covered by a sleeve coupled to the transmitting coupling inductor in a shirt for constant power and monitoring. The sensor platform 100 may be attached anywhere on the wearer's body.

FIG. 2 illustrates an exemplary embodiment to disclosing a fabrication technique of the sensor platform 100, 200 in accordance with implementations of the present disclosure. The fabrication for inductor-capacitor (LC) or resistive-LC (RLC) elements for electronic readouts. A base substrate layer 104 may be formed utilizing a number of different techniques and may be at least partially formed of at least one or more of bamboo, lignin, chitosan, starch, cellulose, etc., which serve as precursors to the LIG elements. For example, a mesh of bamboo yarns, combined with elastomer (PGS - polyglycerol sebacate or other) may be cut to the required shape. The LIG process may be performed on the base substrate layer 104 to create an inductor-capacitor circuit 102 and one or more skin sensing elements 106, along with any electrical vias and traces to connect the electronic circuitry. LIG fabricated vias are formed using the fabrication laser to cut holes in the substrate and subsequently line them with LIG elements 102 and 106 to create an electrical connection between each side of the base substrate layer 104. The base substrate layer 104 may then be coated with a thin encapsulation layer 114 which may be on the order of 0.1˜100 μm thick, the thickness may be tuned to meet any mechanical properties of the base substrate layer 104. The one or more LIG fabricated skin sensing elements 106 may be functionalized for detection, for example with a coating of an enzyme, such as but are not limited to, glucose oxidase (GOx), glucose dehydrogenase (GDH), lactate oxidase, and ascorbate oxidase. In an example, multiple sensors may be placed on the same sensor platform 100, 200 using inductor/capacitor/resistor circuits tuned to different resonant frequencies with at least an order of magnitude spacing between frequencies. The one or more LIG fabricated skin sensing elements 106 may be configured to implement sensing via a resistance change or generation of a voltage in response to biochemical activity. Further sensing elements may also be formed onto the sensor platform 100, 200, such as one or more subcutaneous sensing elements 108 and/or one or more optical sensors 110. Thereafter, starch and/or other adhesives may be patterned on the side of the sensor platform 100, 200 for adhesion to the skin of the wearer. Once applied, such adhesives form an adhesion layer 112. The sensing platform 100, 200 may then be applied to the skin 202 of a wearer.

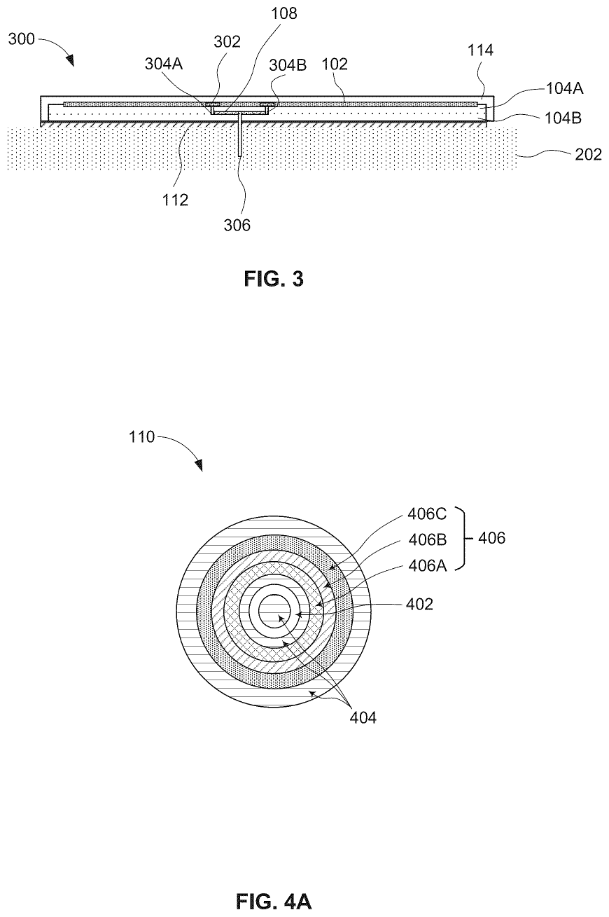

FIG. 3 illustrates an exemplary embodiment of a sensor platform 100, 200, 300 including a subcutaneous sensing element 108, in accordance with implementations of the present disclosure.

In an embodiment, the sensor platform 100, 200, 300 may include one or more subcutaneous sensing elements 108 with a needle 306 to sense biochemical activities of a wearer, one or more LIG fabricated contacts 302, one or more LIG fabricated vias 304A and 304B, and at least a first substrate layer 104A and a second substrate layer 104B forming the base substrate layer 104.

LIG fabricated elements 302 and 304A and 304B on the base substrate layer 104 may be used to electrically connect the one or more subcutaneous sensing elements 108 to the communication and sensing circuitry of the sensor platform 100, 200, 300. The one or more subcutaneous sensing elements 108 may be used to detect one or more biochemical parameters, for example glucose, cortisol, sodium, lactate, ethanol, etc., by inserting a probe of the subcutaneous sensing element 108 beneath the surface of the skin. The subcutaneous sensing element 108 detects a change in a chemical property, such as the concentration of glucose or lactate, and generates a voltage and/or a current in response to the change. The one or more subcutaneous sensing elements 108 may be inserted just below the skin using a minimally invasive procedure by inserting a needle 306 of the subcutaneous sensing element 108 or a small incision. The one or more subcutaneous sensing elements 108 may be configured to detect specific parameters of a wearer, for example, biochemicals such as, glucose, cortisol, etc. For example, the one or more subcutaneous sensing elements 108 may detect glucose levels in an interstitial fluid, that is, the fluid between cells of a wearer. On receiving power, the one or more subcutaneous sensing elements 108 generate electrical signals in response to the detected parameter. For example, a subcutaneous sensing element 108 may apply a constant voltage or current bias to an embedded transistor, and the resistance of the transistor is then measured when the sensor receives the power. These signals are proportional to the concentration of the biochemical being measured. Moreover, the energy generated by the biochemical reaction (either voltage or current) may be detected using a transistor, wherein the resistance across the transistor's two terminals'changes when the generated signal is applied to the base or gate terminal. The nature of the signal source may be used to determine the best transistor type. The resistance change may then be read by the wireless interface in the same manner that a purely resistive sensing element is. The one or more subcutaneous sensing elements 108 may include microneedles and may be combined with optical sensors.

The one or more subcutaneous sensing elements 108 may be integrated to the sensor platform 100, 200, 300 with a moldable and/or curable material, for example, pourable liquid material (e.g., lignin, starch, chitosan, cellulose or other) mixed with at least one elastomer (e.g., polyglycerol sebacate or other) molded, spun coated, deposited, and/or printed to shape as a first layer 104A, at least two hollows are etched to allow at least two vias 304A and 304B to connect the subcutaneous sensing element 108 to the main circuitry. The subcutaneous sensing element 108 may be formed with at least one contact 302, wherein the at least one contact is formed by LIG. Thereafter, a three-terminal apparatus, the transistor, is formed with the LIG and a semiconducting material such as PEDOT-PSS is created. In such configuration, the subcutaneous sensing element 108 may cause a voltage or current bias to the gate of the embedded transistor, and the resistance of the transistor is then measured when the sensor receives the power. LIG may be created on the substrate to form a coupling inductor and transistor terminals along with any traces and vias 304A and 304B. A second substrate layer 104B may be formed by a moldable and/or a curable material, for example, a pourable liquid material, in the same manner as of the first layer 104A. The base substrate layer 104A and 104B forms the base substrate layer 104 which may then be coated with a thin encapsulation layer 114, tuned to target mechanic properties of the sensor platform 100, 200, 300.

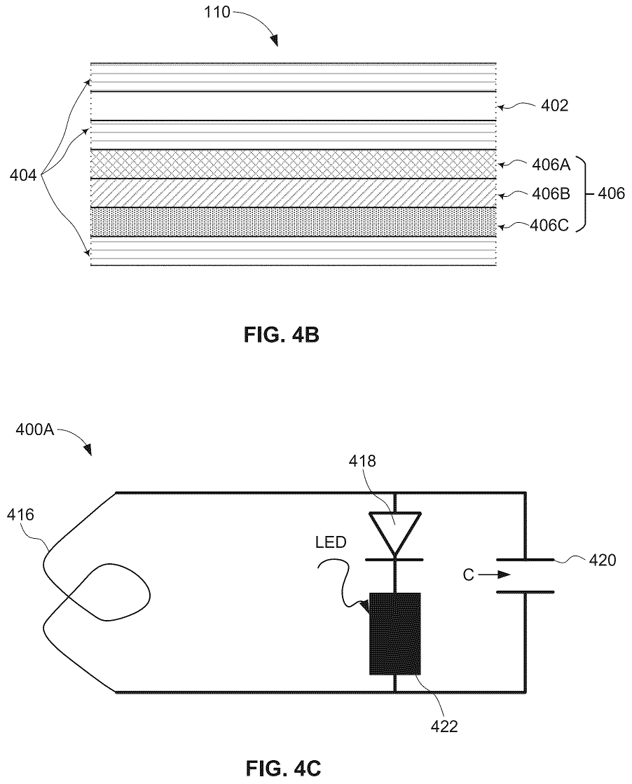

FIG. 4A illustrates an optical sensor 110 which is in the form of an optical emitter-detector ring, in accordance with implementations of the present disclosure. In one embodiment, the optical sensor 110 includes a photodetector 402, one or more conducting contacts 404, and a photoemitter 406. In one embodiment, the photoemitter 406 is made of one or more organic LED layers, for example, layers 406A, 406B, and 406C. The optical sensor 110 may be printed using inkjet printing, direct ink-writing, screen printing, or any other suitable techniques. The optical sensor 110 may be printed with at least one photodetector 402 at the center and at least one photoemitter 406 on the outside. The photodetector 402 and photoemitter 406 may be fabricated in sets, for example in pairs, although any combination may be desirable. The photodetector 402 and photoemitter 406 may be fabricated in a single run, locating the detector 402 at the center of the optical sensor 110. It is important to carefully select the photodetector 402 and photoemitter 406 structures that enables a common electrode material, such as PEDOT-PSS to reduce complexity in the overall design. In one example, the photodetector 402 may be made using Poly(3-hexylthiophene) (P3HT) and the photoemitter 406 may be made using conjugated polymer, for example Poly[2-(4-(30,70-dimethyloctyloxy)-phenyl)-p-phenylene-vinylene] (P-PPV). A translucent material such as rice paper or glass may serve as the substrate for the optical sensor 110. Devices may be transferred post deposition or remain on the initial substrate, for example, translucent biodegradable substrates such as rice paper may also introduce pre-patterned LIG traces beneath the optical components patterned area if this material is incorporated into the final design. The fabrication process disclosed incorporates optical devices that include printing of the optical devices on a translucent substrate and/or transferring one or more optical devices after forming of the base substrate layer. The fabrication disclosed in the present disclosure allows the creation of a multi-layered structure, which is necessary for polymer based optical components. The materials may be deposited using a sequence of prints, starting with the photodetector 402 and adding rings of necessary materials to include one or more conducting contacts 404, the photoemitter 406 with the one or more organic LED layers, for examples layers 406A, 406B, and 406C to create a horizontally structured detector/emitter ring 110. Material patterning may be completed in succession to ensure uniform electrical contact is made between layers or with tuned viscosity that enable continuous horizontal interfaces. Furthermore, the materials such as PEDOT-PSS may be implemented as the one or more conducting contacts 404. A small top contact of PEDOT-PSS or other materials can be added on the photodetector 402 as the photodetector second contact. After deposition of the skin sensor substrate, vias may be filled with PEDOT-PSS (or chosen material) to make contact between the optical components and LC passive circuitry. Using the connection to the optical devices, resistance changes in the photodetector can be detected and read out as a resistive sensor device using the wireless readout. The fabrication of the optical device includes different optical or conducting components like 402, 404, and 406, to be a part of single optical device. The present disclosure discloses fabricating the optical devices including the photoemitter surrounding a photodetector. Further, a biodegradable material may be deposited with respect to the optical devices to form the base substrate layer. The optical devices are formed in a plurality of horizontally structured, contacted concentric rings, in which the photodetector may be located at a center of the one or more optical devices. They are expanded to disclose in the FIG. 4 for the sake of clarity and understanding. The optical sensor and optical devices disclosed in the present disclosure may be biodegradable.

In an exemplary embodiment, the one or more optical sensors 110 can detect biochemical activity of a wearer. In a preferred embodiment, the one or more optical sensors 110 are biodegradable. The one or more optical sensors 110 include one or more photodetectors 402 and one or more photoemitters 406 which may optimally be arranged in pairs depending on their application. The base substrate layer 104 functions as a precursor material for forming electrically conductive pathways in and/or on the base substrate layer 104. For example, but not limited to the electrically conductive pathways that may be formed using laser-induced graphene (LIG). During the fabrication process, optical components may be patterned in a preferrable configuration, for example as concentric circles with an emitter on the outside and a detector on the inside. Other layouts and configurations can be used based on targeted detection, materials, signal strengths and other properties. In some examples, materials such as DNA-based mixtures or polymer LEDs (PLED) can create the emitting and electronic layers of the devices. The one or more optical sensors 110 may also be formed from other organic material. Optical materials may be deposited using printing techniques or through other self-assembly methods. After formation of the optical structure, integration with the substrate may be accomplished by multiple techniques depending on final structure and material required. A process may be disclosed wherein, at least one pourable liquid material (e.g., lignin, starch, chitosan, cellulose or other) mixed with at least one elastomer (e.g., polyglycerol sebacate or other) may be molded, spun, deposited, or printed to a required geometry around the optical sensor 110. A thin layer of substrate material may then be left on top of the one or more optical sensors 110 to create LIG contacts. This process encloses the optical sensor in the substrate with the embedded optics. The LIG process may be performed on the substrate to create electrical couplings between the inductor-capacitor circuit 102 and the one or more optical sensors 110, along with any traces and vias. The base substrate layer 104 may be coated with a thin encapsulation layer 114 which may be hydrophobic, tuned to target mechanic properties of the sensor platform 100, 200, 300. Also, the starch or other adhesive may be patterned on the underside of the sensor patch, around any optical sensors 110.

FIG. 4B illustrates an optical sensor 110 in the form of a stripe, in accordance with an embodiment of the present disclosure. As shown, the photodetector 402, the one or more conducting contacts 404, the photoemitter 406 with the one or more organic LED layers, for examples layers 406A, 406B, and 406C, are formed in the form of stripes.

FIG. 4C illustrates an exemplary embodiment of the electronic circuitry 400A of an optical sensor 110 fabricated on the sensor platform 100, 200, 300, in accordance with implementations of the present disclosure.

In some embodiments, the electronic circuitry 400A of the optical sensor 110 includes at least one inductor coil 416, at least one diode 418, at least one capacitor 420, and at least one light emitting diode 422. In a preferred embodiment, the at least one diode 418 and the at least one light emitting diode 422 are arranged in series; and are in parallel with the at least one capacitor 420 and in parallel with the at least one inductor coil 416. The emitter and detector devices are electrically connected in series to maintain compatibility with the readout circuitry, as the detector resistance decreases, the voltage on the optical emitter increases thereby increasing light output and further decreasing the detector resistance until an equilibrium is reached, this equilibrium state is read as a resistance change along with a small capacitance change that influence the output of the LC passive readout.

FIG. 5A illustrates an exemplary embodiment of a system 500A for monitoring biochemical activity of a wearer, in accordance with implementations of the present disclosure.

The system 500A includes a wearable garment 502, which may be a shirt, the sensor platform 100, 200, 300 which may be included into a bandage 504 and positioned anywhere on a body of the wearer, a battery 506, and a transmitter/receiver device 508 embedded into the wearable garment 502. The wearable garment 502 may further include a transmitter/receiver controller to transmit the wireless signals and receive data associated with the biochemical activity of the wearer, coupled to the transmitter/receiver device 508, and a data storage device 510 to store the received data associated with the biochemical activity. In one example, the transmitter/receiver device 508 is textile-based, including an inductor coil comprising materials such as cotton for a substrate and MXene inks to create the conducting coil, other suitable alternatives may also be used. When in operation, the transmitter/receiver device 508 is positioned to overlap the inductor-capacitor circuit 102 of the sensor platform 100, 200, 300 to inject power into the sensor platform 100, 200, 300 and enable receipt of the electronic state of the sensor platform 100, 200, 300. The transmitter/receiver device 508 may be configured to inject power into the sensor platform 100, 200, 300, and hence to take readings either continuously, or at predefined intervals. Such power injection will result in either a continuous receipt of data from the electronic state of the sensor platform 100, 200, 300, or receipt of data at the predefined intervals. This allows the system 500A to monitor the biochemical activity of the wearer, associated with the electronic state of the sensor platform 100, 200, 300, either continuously, or at predefined intervals (discontinuous mode). In a discontinuous mode, a charge of the battery 506 may be preserved and hence allow for an operation of the device for longer periods. Other wearable wireless transmitters such as flex-circuit or silver-yarn woven devices are also possible.

In an example, the system 500A may be disclosed such that the sensor platform 100, 200, 300, is attached to the body of a wearer to measure the biochemical activity of the wearer. The sensor platform 100, 200, 300 may be attached to an arm, leg, chest, back, or other suitable location on the skin of the wearer. The sensor platform 100, 200, 300 may be configured to monitor specific biochemical activity such as cortisol to track stress of the wearer. To operate the sensor platform, a wearable garment 502 may be worn by the wearer, which may be a shirt. The wearable garment 502 includes a transmitter/receiver 508 and a battery 506. The battery 506 may be a portable battery to provide power to the sensor platform 100, 200, 300 (e.g., a textile energy storage such as MXene supercapacitor, and/or a lithium-ion battery etc.). The wearable garment 502 may include a textile coupling inductor 508 that allows for communication with the inductor-capacitor circuit 102 of the passive wireless sensor platform 100, 200, 300. For example, the sensor platform 100, 200, 300 may be placed on an arm of the wearer and the corresponding transmitter/receiver 508 of the wearable garment 502 may be positioned at a location inside the sleeve of the wearable garment 502 that coincides with the location of the sensor platform 100, 200, 300 on the arm of the wearer. When the transmitter/receiver 508 of the wearable garment 502 is powered on, the transmitter/receiver 508 embedded in the wearable garment 502 induces power to the sensor platform 100, 200, 300 and is able to receive data from the sensor platform 100, 200, 300 which corresponds to the biochemical activity of the wearer. The received data may be recorded by the transmitter/receiver 508 at a data store 510 or transmitted to an external device such as a smart phone for notification and/or display. In an embodiment where the components of the sensor platform 100, 200, 300 are fully biodegradable, when the sensor platform 100, 200, 300 is no longer needed, it may be composted or recycled creating minimal impact to the environment on its disposal. After the disposal of the sensor platform 100, 200, 300 the wearable garment 502 may be used with a different sensor platform 100, 200, 300. The wearable garment 502 disclosed here may provide a basic electronics infrastructure, that is, charging, energy storage and support circuitry, interconnects with conductive threads, etc. The wearable garment 502 may collect signals from multiple sensor platforms 100, 200, 300 located in different areas on the wearer's body. The wearable garment 502 may connect to computing devices, such as a mobile phone for additional analysis and ease of viewing/accessing the data. The embedded electronics in the wearable garment 502 may perform different operations including power regulation, data storage, intelligence, display/alerts, energy storage, control etc. It is important to note that when data is received from the sensor platform 100, the data may be processed by the computing device to analyze the biochemical parameters associated with the wearer, thereby enabling the monitoring of the wearer's biochemical activity.

FIG. 5B discloses a bandage 504 with an integrated sensor platform 100, 200, 300, and a corresponding transmitter/receiver 508 embedded into the wearable garment 502. In this example the sensor platform 100, 200, 300 is co-located with the transmitter/receiver 508 of the wearable garment 502 and with the sensor platform 100, 200, 300 inductively coupled to the transmitter/receiver 508.

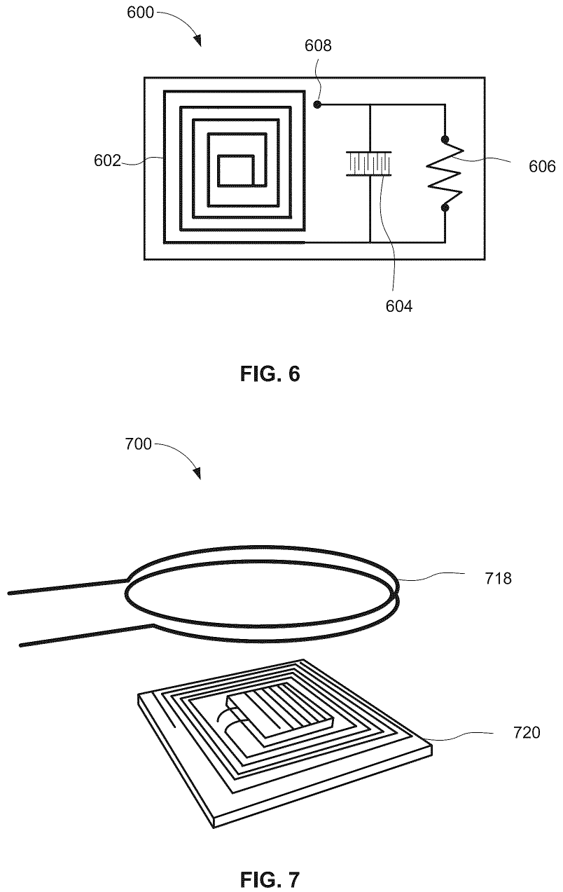

FIG. 6 illustrates an example circuit 600 and may include a receiving coupling inductor and passive components such as a capacitor 604, and a resistor 606.

In accordance with some embodiments of the present disclosure, circuit 600 may be configured to be a sensing circuit of skin sensing element 106. In one embodiment, the capacitor is coated with appropriate enzymes and operates as a sensor, and a resistor 606 with a constant resistance is provided. In some configurations the resistor 606 is merely the internal resistance of the circuit 600 and no separate resistor component is required. In another embodiment, the resistor is coated with appropriate enzymes and operates as a sensor, with the capacitor 604 set to a constant capacitance. The sensor circuit may be formed using LIG on the base substrate 104 of the sensor platform 100, 200, 300. One or more LIG fabricated skin sensing elements 106, and hence circuits 600, may be placed on the same sensor platform 100. A powered transmitter/receiver may be used to communicate with the circuit 600 in the same manner as is used for the sensor platform 100, 200, 300.

In some embodiments, circuit 600 is configured as a reference circuit. In a reference configuration, the circuit 600 may be included to provide reference measurements via a receiving coupling inductor 602 and passive components such as a capacitor 604, and a resistor 606 which may remain unchanged and may be tuned to a specific frequency. In one embodiment, the reference configuration circuit 600 may include only the coupling inductor 602 and the capacitor 604. The reference circuit 600 may be formed using LIG on the base substrate 104 of the sensor platform 100, 200, 300. A powered transmitter/receiver may be used to communicate with the reference configuration circuit 600 in the same manner as is used for the sensor platform 100, 200, 300.

FIG. 7 illustrates an example power and read out apparatus 700 for a wireless power and readout process, in accordance with some implementations of the present disclosure. The power and read out apparatus 700 include a powered transmitter/receiver coil 718 and a corresponding inductor-capacitor circuit 720. The corresponding inductor-capacitor circuit 720, which may be the inductor-capacitor circuit 102 of the sensor platform 100, 200, 300 or coupling inductor 602 of the reference circuit 600. Wireless power and readout enable detection of an electronic state of the inductor-capacitor circuit 102 of the sensor platform 100, 200, 300 or coupling inductor 602 of the reference circuit 600. Consistent with previous examples, the powered transmitter/receiver 718 injects energy via magnetic coupling into the inductor-capacitor circuit 720. The transferred energy may be used to power the passive elements of either the sensor platform 100, 200, 300 or the reference circuit 600. For example, an electronic state of the sensor platform 100, 200, 300 may be communicated to the powered transmitter/receiver 718 via the inductor-capacitor circuit 720 as a function of the input impedance to the coupling with the sensor platform 100, 200, 300. The received signal may be a change in transmitted power, spectral shift or phase shift in the received signal. The signal may be extracted as a change in resonant frequency for capacitor-based sensors. Also, the signal may alternatively be extracted as amplitude modulation for resistive sensors. In this example, the active sensors may use a transistor configuration, applying voltage or current to the gate of the transistor to modulate the transistor's resistance. For optical sensors, the change in resistance is the change in conductivity seen in the optical sensor under illumination.

In wireless sensing using the passive LC sensors, the transmitter/receiver coil 718 may be used to wirelessly detect changes in the sensor's parameters. This allows for the transfer of information without direct electrical connections. Further, the LIG circuitry may be used to create alternate readout differentiation, for instance using a resistive bridge to convert analog voltage to multiple digital bits in a non-wireless fashion.

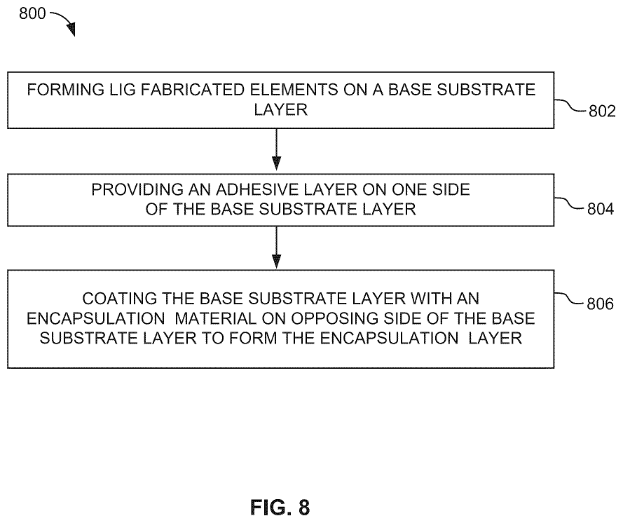

FIG. 8 is a flow diagram illustrating an example method 800 for fabricating the sensor platform, in accordance with an embodiment of the present disclosure. At step 802, the method 800 discloses forming one or more laser induced graphene (LIG) fabricated elements 102 and 106 on the base substrate layer 104. The LIG elements 102 and 106 include an inductor-capacitor circuit 102 to receive a wireless signal and to provide passive wireless readouts, one or more sensing circuits 106 to detect biochemical activity associated with a wearer of the apparatus and one or more connecting traces to electrically couple the inductor-capacitor circuit 102, and the one or more sensing circuits 106.

At step 804, the method 800 discloses providing an adhesive layer 112 on a one side of the base substrate layer 104 to provide adhesion to the skin of a wearer. In one embodiment, starch-based adhesives or similar materials with a thickness in the order of 10s of microns may be used for the adhesion layer 112 to ensure that the sensor platform 100 retains a thin form factor.

At step 806, the method 800 discloses coating the base substrate layer 104 with an encapsulation material to form an encapsulation layer 114 on an opposite side of the base substrate 104. The encapsulation layer 114 offers water resistance and/or provides hydrophobic encapsulation. The encapsulation layer 114 may be fabricated using material such as, but not limited to, shellac, modified starch, modified cellulose, gelatin etc. As described, the adhesive layer 112 enables attachment of the sensor platform 100 to the wearer's skin and the encapsulation layer 114 secures the device from water, dust, external radiations, etc.

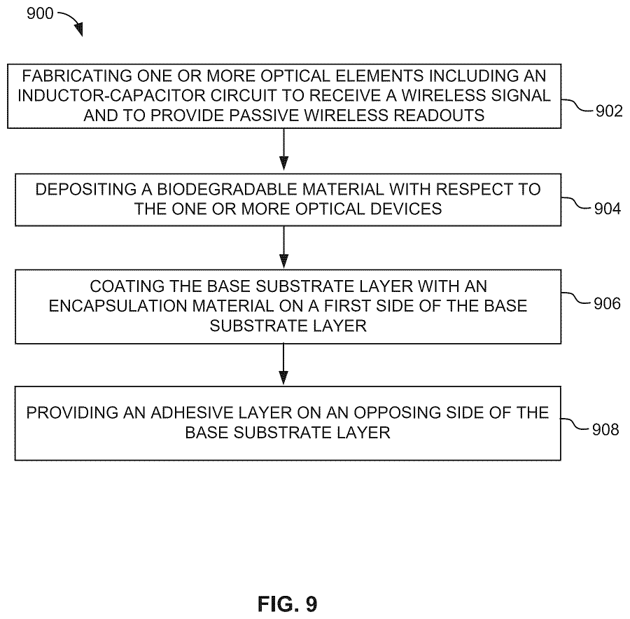

FIG. 9 is a flow diagram that represents an example method 900 for fabricating an apparatus with optical elements, in accordance with an embodiment of the present disclosure. At step 902, the method 900 discloses fabricating one or more optical elements, the one or more optical elements include a photoemitter and a photodetector. That is, the one or more optical elements include an inductor-capacitor circuit to receive a wireless signal and to provide passive wireless readouts. At step 904, the method 900 discloses depositing a biodegradable material with respect to the one or more optical elements to form base optical elements. At step 906, the method 900 discloses coating the base substrate layer with a encapsulation material on a first side of the base substrate layer to form a hydrophobic substrate layer. At step 910, the method 900 discloses providing an adhesive layer on an opposing side of the base substrate layer. The method steps may be described in detail in conjunction with FIG. 5 and FIG. 1.

In view of this, implementations of the present disclosure propose a batteryless biodegradable implantable sensor platform to provide continuous updates to the wearer. These apparatuses 100, 200 and 300 are configured to continuously monitor cortisol or other bio signal levels in the human body and the disclosed apparatuses are sustainable and eco-friendly. The apparatuses are powered wirelessly, such as through electromagnetic waves, magnetic coupling, for monitoring. Thus, the wearer's health and different body parameters can be regularly monitored using a portable apparatus as disclosed in the present disclosure. The apparatus is powered by the wearable components on, for example, a smart shirt that can induce power to the apparatus thereby making the apparatus light weight. The apparatus is lightweight, biocompatible and can easily be worn even by a newborn child. The apparatus being biodegradable produces no toxic waste that would otherwise contribute to landfills and the damage of natural ecosystems.

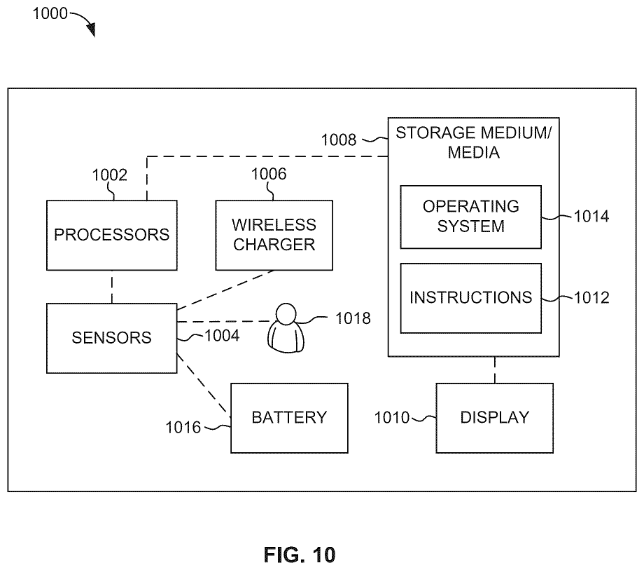

FIG. 10 illustrates a block diagram of an example environment 1000 in accordance with implementations of the present disclosure. The environment includes processors 1002, apparatuses 1004, a wireless charger 1006, a storage medium 1008, a display 1010, a set of instructions 1012, an operating system 1014, a battery 1016 and a wearer 1018. The sensors 1004 for example includes without limitation, a glucose sensor that is responsive to glucose levels in blood. A glucose sensor can be described as an amperometry electrochemical biosensor that generates a current in response to the electrochemical reaction between glucose and a glucose oxidase. In some examples, glucose sensors may include non-enzymatic electrochemical reactions between glucose and inorganic catalysts on electrodes. It is contemplated, however, that the one or more sensors 120 can include any appropriate sensor to sense heart rate, fluid in the body, sweat parameters, stress etc.

The environment may disclose computing machines such as desktops, laptops, smartphones, tablets, and wearables which may be used to store data often have limited storage structure of the computer system. The environment 1000 may include additional components not shown and that some of the process components described may be removed and/or modified. In another example, the environment 1000 may be implemented on external-cloud platforms or internal corporate cloud computing clusters, or organizational computing resources, etc.

The environment 1000 includes processor(s) 1002, such as a central processing unit, ASIC or another type of processing circuit, input/output devices 1010, such as a display, band, tablet, etc., a network interface, such as a Local Area Network (LAN), a wireless 802.11x LAN, a 3G or 4G mobile WAN or a WiMax WAN, and a processor-readable medium. Each of these components may be operatively coupled to a bus or a network or inductive coupling. The computer-readable medium may be any suitable medium that participates in providing instructions to the processor(s) 1002 for execution. For example, the processor-readable medium may be non-transitory or non-volatile medium, such as a magnetic disk or solid-state non-volatile memory or volatile medium such as RAM.

The computer system may include a storage medium/media 1008, which may include non-volatile data storage. The data storage stores any data provided by analyzing the wearer 1018 parameters received from the biochemical sensor system 200. The data storage may be used to store information extracted from the wearer's parameters and from devices/sensors 100, 200 during operation.

The network interface connects the computer system to internal systems for example, via a LAN. Also, the network interface may connect the computer system to the Internet. For example, the computer system may connect to web browsers and other external applications and systems via the network interface to communicate with external devices such as display 1010.

A computer program (also known as a program, software, software application, script, or code) may be written in any appropriate form of programming language, including compiled or interpreted languages, and it may be deployed in any appropriate form, including as a stand-alone program or as a module, component, subroutine, or other unit suitable for use in a computing environment. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

Processors suitable for the execution of a computer program include, by way of example, both general and special purpose microprocessors, and any one or more processors of any appropriate kind of digital computer. Generally, a processor will receive instructions and data from a read-only memory or a random-access memory or both. Elements of a computer can include a processor for performing instructions and one or more memory devices for storing instructions and data. Generally, a computer will also include or be operatively coupled to receive data from or transfer data to, or both, one or more mass storage devices for storing data (e.g., magnetic, magneto optical disks, or optical disks). However, a computer need not have such devices. Moreover, a computer may be embedded in another device (e.g., a mobile telephone, a personal digital assistant (PDA), a mobile audio player, a Global Positioning System (GPS) receiver). Computer readable media suitable for storing computer program instructions and data include all forms of non-volatile memory, media and memory devices, including by way of example semiconductor memory devices (e.g., EPROM, EEPROM, and flash memory devices); magnetic disks (e.g., internal hard disks or removable disks); magneto optical disks; and CD ROM and DVD-ROM disks. The processor and the memory may be supplemented by, or incorporated in, special purpose logic circuitry.

The computing system may include clients and servers. A client and server are generally remote from each other and typically interact through a communication network. The relationship of client and server arises by virtue of computer programs running on the respective computers and having a client-server relationship to each other.

The energy may be stored and then used from the battery 1016 that is used to power components of one or more of the devices of the system 500, the processor, and the communications system on the wearable garment. The battery can include any appropriate ESD or combinations thereof. Example ESDs can include, without limitation, a battery, a capacitor, and/or a supercapacitor. For example, the device 100, 200 can be powered by an ESD that includes a flexible lithium polymer (LiPo) battery, a zinc-ion battery (ZIB), and/or a MXene-based flexible supercapacitor, each of which can be manufactured to be relatively thin, flexible, and stretchable.

While this specification contains many specifics, these should not be construed as limitations on the scope of the disclosure or of what may be claimed, but rather as descriptions of features specific to implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

A number of implementations have been described. Nevertheless, it will be understood that various modifications may be made without departing from the spirit and scope of the disclosure. For example, various forms of the flows shown above may be used, with steps re-ordered, added, or removed. Accordingly, other implementations are within the scope of the following claims.

What has been described and illustrated herein is an example along with some of its variations. The terms, descriptions, and figures used herein are set forth by way of illustration only and are not meant as limitations. Many variations are possible within the spirit and scope of the subject matter, which is intended to be defined by the following claims and their equivalents.

While this specification contains many specifics, these should not be construed as limitations on the scope of the disclosure or of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

A number of implementations have been described. Nevertheless, it will be understood that various modifications may be made without departing from the spirit and scope of the disclosure. For example, various forms of the flows shown above may be used, with steps re-ordered, added, or removed. Accordingly, other implementations are within the scope of the following claims.

What is claimed is:

1. An apparatus for monitoring the biochemical activity of a wearer, comprising:
a base substrate layer comprised of a carbon-based material having one or more laser induced graphene (LIG) fabricated elements, the one or more LIG fabricated elements including:
an inductor-capacitor circuit to receive a wireless signal and to provide passive wireless readouts; and
one or more sensing circuits electrically coupled to the inductor-capacitor circuit to detect biochemical activity associated with the wearer of the apparatus;
an adhesive layer provided on a first side of the base substrate layer to provide adhesion to the skin of the wearer; and
an encapsulation layer placed on an opposing side of the base substrate layer.

2. The apparatus of claim 1, wherein the base substrate layer, the one or more LIG fabricated elements, the adhesive layer, and the encapsulation layer are biodegradable.

3. The apparatus of claim 1, wherein each of the one or more sensing circuits detects a respective biochemical activity of: glucose, cortisol, sodium, lactate, or ethanol.

4. The apparatus of claim 1, wherein the distance between a distal side of the adhesive layer and a distal side of the encapsulation layer is 1 μm to 200 μm.

5. The apparatus of claim 1, wherein the one or more sensing circuits are electrically passive.

6. The apparatus of claim 1, wherein the encapsulation layer is water resistant and/or hydrophobic.

7. The apparatus of claim 1, wherein the one or more LIG fabricated elements further includes a reference receiver circuit to provide a static reference signal to compare with sensed signals.

8. The apparatus of claim 1, further comprising a biodegradable optical sensor to detect the biochemical activity, wherein the biodegradable optical sensor comprises at least one photodetector and at least one photoemitter.

9. The apparatus of claim 1, wherein one of the one or more sensing circuits is coated with an enzymatic material to detect the biochemical activity.

10. The apparatus of claim 1, further comprising a biodegradable subcutaneous sensor to perform a subcutaneous interrogation.

11. The apparatus of claim 1, wherein the one or more sensing circuits are configured to implement resistive or capacitive sensing.

12. The apparatus of claim 1, wherein the one or more sensing circuits are configured to implement active sensing via generation of a voltage responsive to the biochemical activity.

13. A system comprising:
an apparatus for monitoring biochemical activity of a wearer, comprising:
a base substrate layer comprised of a carbon-based material having one or more laser induced graphene (LIG) fabricated elements, the one or more LIG fabricated elements including:
an inductor-capacitor circuit to receive a wireless signal and to provide passive wireless readouts; and
one or more sensing circuits electrically coupled to the inductor-capacitor circuit to detect biochemical activity associated with the wearer of the apparatus;
an adhesive layer provided on a first side of the base substrate layer to provide adhesion to the skin of the wearer; and
an encapsulation layer placed on an opposing side of the base substrate layer, and
a transmitter receiver device, the transmitter receiver device comprising:
a transmitting controller to transmit the wireless signals and receive data associated with the biochemical activity;
data storage to store the data associated with the biochemical activity; and
a battery to provide and regulate power for the biochemical sensor system.

14. The system of claim 13, wherein the transmitter/receiver device comprises a textile-based receiver element to receive the wireless signals.

15. The system of claim 13, wherein the transmitter receiver device induces electrical energy into the inductor-capacitor circuit via a magnetic coupling.

16. A method for fabricating an apparatus for monitoring biochemical activity of a wearer, the method comprising:
forming one or more laser induced graphene (LIG) fabricated elements on a base substrate layer, the one or more LIG fabricated elements including:
an inductor-capacitor circuit to receive a wireless signal and to provide passive wireless readouts;
one or more sensing circuits electrically coupled to the inductor-capacitor circuit to detect biochemical activity associated with the wearer of the biodegradable sensor;
providing an adhesive layer on a first side of the base substrate layer to provide adhesion to the skin of the wearer; and
coating the base substrate layer with an encapsulation material on an opposing side of the base substrate layer to form an encapsulation layer.

17. The method of claim 16, wherein providing the base substrate layer includes adding an elastomer material to increase flexibility of the base substrate layer.

18. The method of claim 15, further comprising:
fabricating one or more optical devices, the one or more optical devices including a photoemitter surrounding a photodetector; and
depositing a biodegradable material with respect to the one or more optical devices to form the base substrate layer.

19. The method of claim 18, wherein the one or more optical devices are formed in a plurality of horizontally structured, contacted concentric rings, and wherein the photodetector is located at a center of the one or more optical devices.

20. The method of claim 18, wherein fabricating the one or more optical devices includes printing the one or more optical devices on a translucent substrate and lifting the one or more optical devices after forming of the base substrate layer.
